SEQUENCE LISTING (12 sequences)

SEQ ID NO: 1 - protein, 12 aa, Artificial
  derived from Homo sapiens
CXCXXXXXXCXC

SEQ ID NO: 2 - protein, 38 aa, Artificial
  derived from Homo sapiens
ATLTQEDIQQIMKQLNKKEPVETIECNCIPGQCECKKQ

SEQ ID NO: 3 - protein, 38 aa, Artificial
  derived from Homo sapiens
LSQTDILQLQQLLTAKAATAPEKVACDCLPNKCDCEKE

SEQ ID NO: 4 - protein, 38 aa, Artificial
  derived from Homo sapiens
MTLSDIEKLEALLLSKKANAVPEVKCNCIVGQCSCYEH

SEQ ID NO: 5 - protein, 38 aa, Artificial
  derived from Homo sapiens
SLTEQVSRLDIDDNDMGLGGSETMECKCGMPLCICVAP

SEQ ID NO: 6 - protein, 38 aa, Artificial
  derived from Homo sapiens
LSLGKEDDSSPMKNSTVQSTAALIECKCGMPLCICEAP

SEQ ID NO: 7 - protein, 38 aa, Artificial
  derived from Homo sapiens
LNLGEDDASSPMKNSAFHSAPAVIECKCGMPLCICEAP

SEQ ID NO: 8 - protein, 38 aa, Artificial
  derived from Homo sapiens
LYGDAEKPAESGGSQPPRAAARKAACACDQKPCSCSKV

SEQ ID NO: 9 - protein, 38 aa, Artificial
  derived from Homo sapiens
LYGDSGKPAEGGGSVTSRAATGKVACTCDQKPCNCPKG

SEQ ID NO: 10 - protein, 38 aa, Artificial
  derived from Homo sapiens
VPQASLKASDLSDFQSVSKLNQGKPCTCIGKECQCKRW

SEQ ID NO: 11 - protein, 38 aa, Artificial
  derived from Homo sapiens
VSPESLTANDLSDFQSVSKLSQGKPCVCVGKACQCKRW

SEQ ID NO: 12 - protein, 38 aa, Artificial
  derived from Homo sapiens
ASPASLTASQPSDFQSVSILGTAAECTCDEKECQCKNC

(19) United States
(12) Patent Application Publication
(10) Pub. No.: US 2009/0093614 A1
(43) Pub. Date: Apr. 9, 2009

(54) SELF-ASSEMBLED PROTEINS AND RELATED METHODS

(76) Inventor: Charles Dameron, Pittsburgh, PA (US)

Correspondence Address: ECKERT SEAMANS CHERIN & MELLOTT, 600 GRANT STREET, 44TH FLOOR, PITTSBURGH, PA, 15219, US

(21) Appl. No.: 11/751,850

(22) Filed: May 22, 2007

Related U.S. Application Data
(60) Provisional application No. 60/808,232, filed on May 24, 2006.

(51) Int. Cl. C07K 14/00; C07K 1/00

(52) U.S. Cl. 530/324; 530/333

(57) ABSTRACT

The present invention provides user-directed construction of novel specific homo- and hetero-dimeric, and multimeric assemblages of proteins. The present invention is comprised of a family of peptide sequences that forms links between proteins, where the peptide sequences produce a hook or loop which supports specific self-assembly of dimers of the proteins to which they are attached. The hook or loop may have a short aliphatic repeat and a metal binding loop. Also provided is a method of constructing a hook motif of metal binding loop sequences attached to at least one aliphatic carbon sequence to produce homo-dimeric, hetero-dimeric and multimeric assemblages of proteins.

The present application claims priority to U.S. Provisional Application No. 60/808,232, filed May 24, 2006, which is incorporated herein by reference.

### BACKGROUND OF THE INVENTION

1. Field of the Invention

The present invention relates to protein self-assembly methods and, more particularly, to homo- and hetero-dimeric and multimeric assemblages of proteins and methods for their construction.

2. Description of the Prior Art

Conventional technology for creating dimers or multimers from proteins which normally are not coupled requires that the proteins be (1) chemically cross-linked, (2) created as direct gene fusions of the proteins involved or (3) indirectly linked by creating gene fusions with specific protein-protein binding motifs so that they, when translated, would be expected to specifically bind to each other through the added motifs with or without the aid of an exogenous regulatory ligand.

Generally, chemical cross-linking requires that the proteins be isolated and either (1) mixed together and treated with reagents to cause them to be covalently attached to each other or (2) each isolated protein be modified with different reagents that will enable the modified proteins to interact when mixed. In the former case, it is difficult to specifically dimerize the proteins into a single or limited series of structures because the crosslinks can form at multiple locations on the surfaces of the two proteins. In the latter case, each protein is modified separately with compounds that will couple with the other when mixed and thereby link the two proteins. Neither of these methods produces entirely specific quaternary structural links between the proteins. Both can lead to conformational changes in the proteins being modified and thus perturb their normal function and in some cases cause the complexes to be poorly soluble. Chemically cross-linked proteins are, however, commonly used in in vivo applications by first isolating the proteins, chemically cross-linking them and then injecting them into an organism. Chemical cross-links frequently are used to link enzymes to antibodies, which subsequently are used analytically in ELISA, tissue fixing or other in vitro analyses.

Chemical cross-linkers capable of diffusing across cell membranes have been used to study the state of oligomerization of dimeric and hexameric species and to probe for heterocomplexes. These studies are best performed on well characterized proteins so that appropriate linkers can be used. The weakness of these methods is that it is not possible to entirely limit the modification to only those proteins being targeted.

Though there are in vitro cleavable chemical cross-linkers, equilibrium between the monomer and dimer cannot be maintained, manipulated or used as a switch. Few of the modifying species can serve as a tool for subsequent affinity purification. Biotinylation, which has been an important tool for in vitro affinity purification (Pierce), cannot be used to purposely construct dimers or higher order species in vivo.

Pierce Biotechnology Inc. markets a wide range of chemical cross-linkers and bioconjugate linkers that interact with a variety of functional groups such as amino, sulphydryls and carboxylates. Some of these cross-linkers diffuse across membranes, but their reactions are largely non-specific and target a broad number of proteins within a cell.

Directly fusing two genes so that they are expressed as one fusion protein is commonly used to add an affinity tag to a protein. The affinity tag then enables the protein fusion to be purified more easily or, in some cases, used as an analytical tool to detect or measure the protein. Proteins, such as glutathione transferase (Pharmacia) and maltose binding protein (NEB); protein domains, such as S-tag (Novagen) and FLAG (Kodak); and 6 histidine repeats (His tag) frequently are used for affinity purification purposes. Genes or gene fragments also are used to target the fusion protein to specific cellular locations. Novagen (and others) markets a vector that produces a gene fusion of the protein of interest to an export sequence so that the nascent protein will be excreted into the periplasmic space.

The S-tag, FLAG and, especially, the His tag are not as prone to cause folding problems in their fusion partners. However, it is relatively common for the resultant large glutathione transferase and maltose binding protein fusions to have solubility and expression problems. The His tag, which is the most commonly used affinity purification tag on the market today, it the most advantageous affinity tag because it works through a coordination complex and can be used in denaturing conditions. However, although very useful for affinity methods, all of these methods do not enable the construction of homodimers or specific heterospecies.

The direct fusion of the domains derived from the FKDP protein to the proteins of interest can be used to produce homodimers (ARIAD Pharmaceuticals, Inc.) under the control of exogenous rapamycin or related compounds due to the FKDP protein's high affinity for rapamycin. Two FKDP domains bind to each molecule (ligand) or rapamycin. Similarly, using hetero-fusions to FKDP and FRB, domain-specific heterodimers can be induced to form through the addition of a modified rapamycin (ARIAD Pharmaceuticals, Inc.). The regulated feature of these systems is useful and has been used in trafficking studies and may find uses in drug delivery systems. The size and complexity of the fusion proteins, however, pose problems in some cases. The delivery of rapamycin to the cell, tissue or organisms can be difficult. As designed, the monomer to dimer affinity cannot be modified.

There are several cloning systems, so called two-hybrid systems, where the possibility of protein-protein interactions between heteromers can be probed through the formation of dimeric species (Invitrogen and others). The two-hybrid systems also can be used to explore the interactions between specific dimers.

While these systems excel at probing for interactions between one protein and a library of others, they do not enable the formation of homo-dimers of a protein or hetero-dimer formation between proteins selected by the user. The system is, by its nature, linked to a reporter system. In addition, the two-hybrid systems cannot be used to build higher order species, establish an equlibria between the monomer and dimer, etc., or be regulated and used in trafficking studies.

The fusion of genes to a gene or gene fragment, as proposed for the hook motif, enables the formation of homo-dimers in vivo. The possibility of producing fusions of this type has been explored through manipulation of classical leucine-zippers and zinc-fingers. Both of these motifs require a larger sequence be attached to the desired proteins. Both the zipper and finger motifs cannot have their monomer-dimer equilibrium easily manipulated, be used as a switch, be used readily in affinity purification strategies or be used easily to make discrete multimer complexes. To the best of the inventor's knowledge, neither the leucine-zipper nor the zinc finger motifs has been utilized in commercial applications, although the leucine-zipper motif has been explored by an industry group.

With respect to the direct purification of fusion proteins by affinity methods, there are many choices. His-tag (Pharmacia, Sigma and others), S-tag (Novagen), FLAG (Kodak), GST-tag (Pharmacia and others) and maltose binding protein tags all are used in gene fusion based systems to aid in the purification of bacterial and yeast over-expression systems, but these systems are not used in vivo or in vitro to construct dimer or higher order structures and for the most part they are not suited to that task.

There exists a need, therefore, for a method to link proteins to form specific hetero- and homo-dimeric and multimeric protein structures in the living body in vivo and in a test tube or apparatus in vitro as well as for sequence (“hook”) motifs useful as linkers.

### SUMMARY OF THE INVENTION

It is an object of this invention to provide a method of linking proteins without conformational changes in the proteins.

It is another object of this invention to provide a method of linking proteins that enables the construction of homodimers of proteins or construction of heterodimers between proteins.

The above needs are met and objects accomplished by providing a genetic sequence for a peptide sequence, said peptide sequence comprising at least one component comprised of a CxCxxxxCxC-metal binding loop and aliphatic repeat groups, wherein the peptide sequence produces peptides or proteins. The peptides or proteins produced therefrom form homo-dimers, hetero-dimers and multimers of proteins both in vivo and in vitro. The peptide sequence comprising at least one component comprised of a CxCxxxxCxC-metal binding loop and aliphatic repeat groups can include, for example, the following sequence:

ATLTQEDIQQIMKQLNKKEPVETIECNCIPGQCECKKQ

The present invention also provides a method of constructing assemblages of proteins with linking between the proteins. The method is comprised of forming a hydrophilic sequence (“hook”) motif, comprised of a metal binding loop sequence attached to at least one aliphatic carbon sequence; providing a plurality of proteins; and mixing the plurality of proteins so that they self-assemble in the presence of the metal binding loop in order to form protein structures, such as, without limitation, homo-dimers, hetero-dimers or multimers.

The attachment of the metal binding loop sequence to the at least one aliphatic carbon sequence allows for the formation of specific interactions of the plurality of proteins. The aliphatic carbon sequence contains residues which contribute to the specific interaction of the plurality of proteins.

The metal of the metal binding loop is, for example and without limitation, zinc [Zn(II)] or copper [Cu(I)].

The homo-dimers, hetero-dimers and multimers are effective for both in vivo and in vitro uses.

The present invention further provides protein structures produced by the methods of the present invention, in which the protein structures are characterized as being suitable for use for, without limitation, diagnostic reagents; biomarkers; metal-activated switches; cell-trafficking studies; affinity purification of in vivo constructs; nanoscale construction; for imaging of cells and tissues such as, without limitation, visible imaging, fluorescent imaging and confocal imaging; delivery of proteins or pharmaceuticals having said proteins bound thereto to specific tissues or cells; purifying and separating compounds; cell research; and discovery of compounds for treating disease.

### BRIEF DESCRIPTION OF THE DRAWINGS

A full understanding of the invention can be gained from the following description of the preferred embodiments when read in conjunction with the accompanying drawings in which:

FIG. 1 shows the hook motif according to embodiments of the present invention;

FIG. 2 shows realized and potential hook motifs according to embodiments of the present invention;

FIG. 3 shows the hook motif according to embodiments of the present invention, in which (A) contains a CxCxxxxCxC motif and an aliphatic repeat; (B) shows fusion proteins constructed to express the motif; and (C) shows proteins expressing the motif and connecting to make dimers;

FIG. 4 shows hook motifs attached to solid resins for capturing proteins expressing the motifs according to the embodiments of the present invention; and

FIG. 5 shows motifs with varying affinities, in which (A) shows a hetero-trimer constructed with fusions containing four distinct hook motifs; and (B) shows a metal binding loop with an attached N-terminal fluorescent tag used as a probe.

### DESCRIPTION OF THE PREFERRED EMBODIMENTS

The present invention provides homo- and -hetero-dimeric and multimeric self-assembly of proteins and methods for their construction.

In an embodiment of the present invention, there is provided a genetic sequence for a peptide sequence, said peptide sequence comprising at least one component comprised of a CxCxxxxCxC-metal binding loop and aliphatic repeat groups, wherein the peptide sequence produces peptides or proteins. The peptides or proteins produced therefrom form homo-dimers, hetero-dimers and multimers of proteins both in vivo and in vitro. The peptide sequence comprising at least one component comprised of a CxCxxxxCxC-metal binding loop and aliphatic repeat groups can include, for example, the following sequence:

ATLTQEDIQQIMKQLNKKEPVETIECNCIPGQCECKKQ

In another embodiment of the present invention, there is provided a method of constructing assemblages of proteins with linking between the proteins. The method is comprised of forming a hydrophilic sequence (“hook”) motif, comprised of a metal binding loop sequence attached to at least one aliphatic carbon sequence; providing a plurality of proteins; and mixing the plurality of proteins so that they self-assemble in the presence of the metal binding loop in order to form protein structures, such as, without limitation, homo-dimers, hetero-dimers or multimers.

The attachment of the metal binding loop sequence to the at least one aliphatic carbon sequence allows for the formation of specific interactions of the plurality of proteins. The aliphatic carbon sequence contains residues which contribute to the specific interaction of the plurality of proteins.

The metal of the metal binding loop is, for example and without limitation, zinc [Zn(II)] or copper [Cu(I)].

The homo-dimers, hetero-dimers and multimers are effective for both in vivo and in vitro uses.

In a further embodiment of the present invention, there is provided protein structures produced by the methods of the present invention, in which the protein structures are characterized as being suitable for use for, without limitation, diagnostic reagents; biomarkers; metal-activated switches; cell-trafficking studies; affinity purification of in vivo constructs; nanoscale construction; for imaging of cells and tissues such as, without limitation, visible imaging, fluorescent imaging and confocal imaging; delivery of proteins or pharmaceuticals having said proteins bound thereto to specific tissues or cells; purifying and separating compounds; cell research; and discovery of compounds for treating disease.

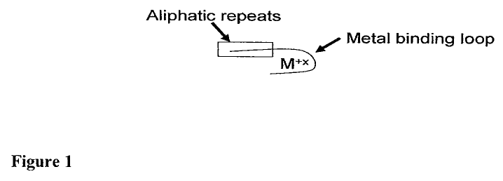

The peptide sequence produces a hook, which supports the specific self-assembly of dimers of the proteins to which they are attached. As shown in FIG. 1, the hook motif consists of a short aliphatic repeat and a metal binding loop. The metal binding motif contains CxCxxxxCxC-sequences. The metal binding loop or hook, utilizes four thiolates in the -CxCxxxxCxC-motif, wherein x is a residue to chelate metal ions such as Zn(II) or Cu(I). The metal binding loop is important for specific interactions between proteins but also aides in the stabilization of the dimer.

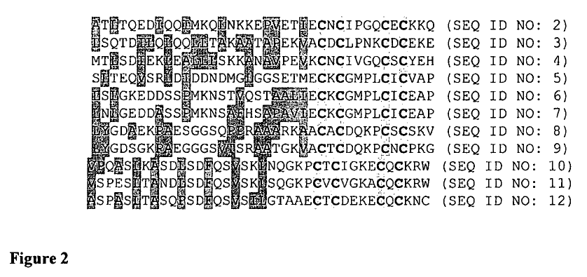

Hook motifs are shown in FIG. 2. Residues around cysteines are responsible for the specificity of the interactions. The top sequence in FIG. 2 has been fully tested and the highlighted portion shows hydrophobic residues, frequently as aliphatic repeats. The cysteines in the metal binding motif are in bold. Four of the genes containing these sequences have been cloned.

The aliphatic repeats support dimerization even in the absence of the metal binding loop, constituting the bulk of the stabilization. However, in the absence of a metal binding loop, there is a higher probability of forming non-specific interactions and non-specific hetero-dimers and multimers. The residues around the aliphatic repeats also contribute to the specificity. Both parts of the peptide sequence are involved in the specificity and stabilization of the protein-protein interactions. To construct dimers or multimers, the DNA sequence that codes for the peptides must be fused to the gene or genes of interest at one of the two termini or, possibly, between two domains. Homo-dimers are formed by fusing a single DNA sequence coding for a peptide motif to the gene of interest. The resultant protein will self assemble into a specific dimer in the presence of zinc(II) or copper(I). Formation of specific hetero species requires that two different motifs, which have a higher affinity for each other than for themselves, be used with the genes of interest. In the absence of metal ions, the motif is prone to more non-specific interactions with similar aliphatic motifs to make dimers or multimers. Most cells maintain an intracellular zinc concentration more than sufficient to insure that zinc is bound to the sequence FIG. 2 shows only a partial selection of the sequences that can be used for these operations according to the embodiments of the present invention. It is envisioned that optimized sequences are used for the specific uses of the sequences.

The advantageous features provided by the metal binding peptides (i.e., the “sequence motif” or “hook motif”) of the present invention are: (1) the sequences are short, and thus they are less likely to interfere with the folding of their fusion partners; (2) the sequences are hydrophilic, and thus they aid, not decrease, the solubility of the fusions; (3) the sequences can be used to form dimers and/or multimers for in vivo and in vitro applications; (4) it is possible to select a range of dissociation constants by varying the length of the aliphatic repeat region; (5) it is possible to build a metal-activated dimer (i.e., metal ions are easily administered); (6) due to the variety of sequences available, a large number of distinct constructs can be produced; (7) the sequences can be used as an affinity tag with similarly tagged proteins, in which the tag then can be used to purify or detect fusion proteins.

The most advantageous feature of the hook motif is the ability to use it to form specific user-directed self-assembling hetero-dimers in vivo. In natural settings, the motif aids in or supports dimerization. The hook motif is found at the termini of proteins and in separate regions or domains of proteins and can, in principle, be utilized in similar positions when used to make novel dimers.

Specific dimerization or the formation of higher order structures is difficult in that it requires the use of large scale structures to interface with one another. One benefit of the sequence motif of the present invention is that it is small and hydrophilic and, therefore, is less likely to interfere with the structural fold and function of the proteins being dimerized. In this case, the dimerization motif is small and the interaction is weakened by the removal of the metal ion. The opportunity to use this feature in living cells or in the construction of nanoscale fabrications outside of cells is unique. The potential to use the metal-binding-dimerization motif in the direct purification of the fusion proteins also is a significant benefit.

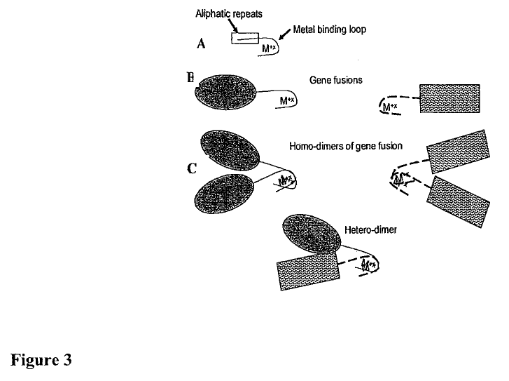

Examples of various ways that the hook motif of the present invention can be employed are shown in FIGS. 3, 4 and 5.

In FIG. 3A, the hook motif is shown to contain a CxCxxxxCxC motif and an aliphatic repeat. FIG. 3B shows fusion proteins constructed to express the motif. FIG. 3C shows proteins expressing the motif and connecting to make dimers. Any proteins expressing the hook motif can participate in the dimer interaction.

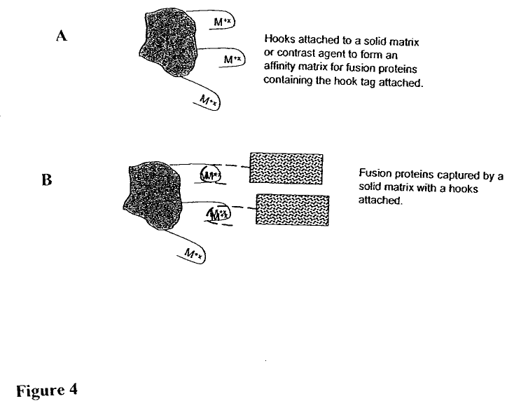

Hook motifs attached to solid resins can be utilized to capture proteins expressing the hook motifs. For example, in FIG. 4A, hooks are attached to a solid matrix or contrast agent to form an affinity matrix for fusion proteins containing the hook tag attached. In FIG. 4B, fusion proteins are captured by a solid matrix with hooks attached.

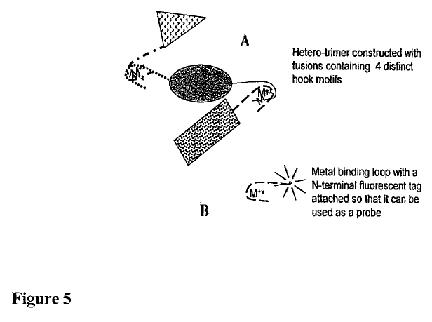

FIG. 5A shows a hetero-trimer fused protein constructed with fusions containing four distinct hook motifs. Thus, the construction of motifs with varying affinities enables specific higher order structures to be built. FIG. 5B shows a metal binding loop with an N-terminal fluorescent tag attached so that it can be used as a probe. In this way, dye molecules attached to a motif can bind to other molecules that contain the hook motif for labeling, cellular trafficking or delivery studies.

The dimers and multimers of the present invention are effective for applications which include, for example and without limitation, cell research; for the delivery of proteins or pharmaceuticals to specific tissues or cells; to discover compounds for treating disease; in cells and tissues for purposes such as, without limitation, visible imaging, fluorescent imaging or confocal imaging; for purification and separation of compounds; for use as a diagnostic reagent; for the self-assembly construction of dimer or higher order protein structures in cells for pharmaceutical applications; as biomarkers; for nanoscale construction; as metal activated switches; in trafficking studies and for affinity purification of in vivo constructs.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1. A peptide sequence, comprising at least one component comprised of a CxCxxxxCxC (SEQ ID NO: 1)-metal binding loop and aliphatic repeat groups, wherein said peptide sequence produces peptides or proteins.

2. The peptide sequence according to claim 1, wherein said peptide sequence comprises ATLTQEDIQQIMKQLNKKEPVETIECNCIPGQCECKKQ (SEQ ID NO: 2).

3. The peptide sequence according to claim 1, wherein said peptides or proteins produced therefrom form homo-dimers of proteins in vivo.

4. The peptide sequence according to claim 1, wherein said peptides or proteins produced therefrom form homo-dimers of proteins in vitro.

5. The peptide sequence according to claim 1, wherein said peptides or proteins produced therefrom form hetero-dimers of proteins in vivo.

6. The peptide sequence according to claim 1, wherein said peptides or proteins produced therefrom form hetero-dimers of proteins in vitro.

7. The peptide sequence according to claim 1, wherein said peptides or proteins produced therefrom form multimers of proteins in vivo.

8. The peptide sequence according to claim 1, wherein said peptides or proteins produced therefrom form multimers of proteins in vitro.

9. A method of constructing assemblages of proteins with linking between the proteins, comprising:
1) forming a hook motif comprised of a metal binding loop sequence attached to at least one aliphatic repeat sequence;
2) providing a plurality of proteins; and
3) mixing the plurality of proteins so that they self-assemble in the presence of the metal binding loop in order to form at least one protein structure selected from the group consisting of homo-dimers, hetero-dimers and multimers.

10. The method according to claim 9, wherein the metal binding loop sequence attached to the at least one aliphatic repeat sequence allows for the formation of specific interactions of the plurality of proteins.

11. The method according to claim 10, wherein the aliphatic sequence contains residues which contribute to the specific interaction of the plurality of proteins.

12. The method according to claim 9, wherein said homo-dimeric protein structures are effective for in vivo uses.

13. The method according to claim 9, wherein said homo-dimeric protein structures are effective for in vitro uses.

14. The method according to claim 9, wherein said hetero-dimeric protein structures are effective for in vivo uses.

15. The method according to claim 9, wherein said hetero-dimeric protein structures are effective for in vitro uses.

16. The method according to claim 9, wherein said multimeric protein structures are effective for in vivo uses.

17. The method according to claim 9, wherein said multimeric protein structures are effective for in vitro uses.

18. The method according to claim 9, wherein the metal is selected from the group consisting of zinc [Zn(II)] and copper [Cu(I)].

19. The method according to claim 9, wherein the hook motif is hydrophilic.

20. Protein structures produced by the method according to claim 9, wherein said protein structures are characterized by being suitable for use as a diagnostic reagent.

21. Protein structures produced by the method according to claim 9, wherein said protein structures are characterized by being suitable for use as biomarkers.

22. Protein structures produced by the method according to claim 9, wherein said protein structures are characterized by being suitable for use as metal-activated switches.

23. Protein structures produced by the method according to claim 9, wherein said protein structures are characterized by being suitable for use in cell-trafficking studies.

24. Protein structures produced by the method according to claim 9, wherein said protein structures are characterized by being suitable for use for affinity purification of in vivo constructs.

25. Protein structures produced by the method according to claim 9, wherein said protein structures are characterized by being suitable for use in nanoscale construction.

26. Protein structures produced by the method according to claim 9, wherein said protein structures are characterized by being suitable for use in cells and tissues for the purpose selected from the group consisting of visible imaging, fluorescent imaging and confocal imaging.

27. Protein structures produced by the method according to claim 9, wherein said protein structures are characterized by being suitable for use for delivery of said proteins structures or pharmaceuticals having said protein structures bound thereto to specific tissues or cells.

28. Protein structures produced by the method according to claim 9, wherein said protein structures are characterized by being suitable for use in purifying and separating compounds.

29. Protein structures produced by the method according to claim 9, wherein said protein structures are characterized by being suitable for use in cell research.

30. Protein structures produced by the method according to claim 9, wherein said protein structures are characterized by being suitable for use for discovering a compound for treating a disease.
